(19) United States
(12) Patent Application Publication
(10) Pub. No.: US 2011/0237781 A1
(43) Pub. Date: Sep. 29, 2011

(54) METHOD OF PREPARING ALPHA-1 PROTEINASE INHIBITOR

(75) Inventors: Wytold Lebing, Seattle, WA (US); Scott A. Cook, Garner, NC (US); Christopher A. Dadd, Holy Springs, NC (US)

(73) Assignee: Talecris Biotherapeutics, Inc., Research Triangle Park, NC (US)

(21) Appl. No.: 13/002,429

(22) PCT Filed: Jul. 17, 2009
(86) PCT No.: PCT/US2009/050982

§ 371 Date: May 31, 2011

Related U.S. Application Data
(60) Provisional application No. 61/081,907, filed on Jul. 18, 2008.

(51) Int. Cl. C07K 1/36

(52) U.S. Cl. 530/395

(57) ABSTRACT

Purification of α-1 proteinase inhibitor (α-1 PI) from solutions comprising α-1 PI is accomplished using hydrophobic interaction chromatography (HIC). In some embodiments, purification of α-1 PI is accomplished by precipitation of contaminating proteins from a starting solution comprising α-1 PI, such as human plasma, followed by anion exchange resin chromatography prior to HIC. Further purification may be accomplished by an optional cation exchange chromatography subsequent to anion exchange chromatography but prior to HIC. Some embodiments of the invention also include virus removal and/or inactivation by methods such as nano filtration and such as contact with a non-ionic detergent. The methods of the present invention result in greater yield, purity, and pathogenic clearance of plasma fractions than known methods.

### CROSS REFERENCE TO RELATED APPLICATION

The present application claims priority to U.S. Provisional Application No. 61/081,907, filed on Jul. 18, 2008 which is herein incorporated by reference in its entirety.

### FIELD OF THE INVENTION

A method for purifying alpha-1 proteinase inhibitor (α-1 PI) is provided using chromatography including hydrophobic interaction chromatography (HIC). In this method, a solution comprising α-1 PI is subjected to hydrophobic interaction chromatography (HIC). The HIC can be preceded and/or followed by one or more other purification steps.

### BACKGROUND OF THE INVENTION

α-1 PI is a glycoprotein with a molecular weight of about 55,000 Daltons. The protein is a single polypeptide chain to which several oligosaccharide units are covalently bound. α-1 PI acts as an inhibitor of endogenous proteases, such as trypsin, chymotrypsin, pancreatic elastase, skin collagenase, renin, urokinase and proteases of polymorphonuclear lymphocytes.

α-1 PI is currently used therapeutically to treat persons having a genetically caused deficiency of α-1 PI. In such a condition, α-1 PI is administered to inhibit lymphocyte elastase in the lungs. Lymphocyte elastase breaks down foreign proteins in the lungs. When α-1 PI is not present in sufficient quantities to regulate elastase activity, the elastase breaks down lung tissue. In time, this imbalance results in chronic lung tissue damage and emphysema. α-1 PI has been successfully used to treat this form of emphysema.

The demand for α-1 PI typically exceeds the available supply. α-1 PI for therapeutic use is currently purified from human plasma. This source of the protein is limited, which contributes to the low supply. In order to maximize the available supply of α-1 PI, a process for purifying α-1 PI from human plasma should have the highest possible yield. The purity of the α-1 PI isolated from human plasma is also critical, because trace impurities can stimulate immune responses in patients who are receiving α-1 PI. Finally, the process of purifying α-1 PI from human plasma using current techniques requires an extensive amount of time, for the separation of the α-1 PI from other proteins, viruses, etc. All of these factors (i.e., low yields, long production times, and low purity), contribute to the inadequate supply of α-1 PI.

An efficient industrial scale method for the purification of α-1 PI that improves the yield and purity of the α-1 PI is needed.

### SUMMARY OF THE INVENTION

In one aspect, the present invention provides a method of purifying alpha-1 proteinase inhibitor in an aqueous solution comprising alpha-1 proteinase inhibitor and other proteins. The method comprises:

a) adjusting the pH, ionic strength, and protein concentration of the aqueous solution so that active alpha-1 proteinase inhibitor does not bind to an ion exchange resin but other proteins in the solution do bind;

b) passing the solution through the ion exchange resin and collecting a flow-through that contains alpha-1 proteinase inhibitor; and

c) contacting the flow-through of step b) with a hydrophobic adsorbent of at least one HIC medium.

In another aspect, the present invention provides a method of purifying alpha-1 proteinase inhibitor from an aqueous solution containing alpha-1 proteinase inhibitor, the method comprising:

a) removing a portion of contaminating proteins from the aqueous solution by precipitation in order to obtain a purified solution containing alpha-1 proteinase inhibitor;

b) passing the purified solution through an anion exchange resin so that alpha-1 proteinase inhibitor binds to the anion exchange resin;

c) eluting alpha-1 proteinase inhibitor from the anion exchange resin to obtain an eluted solution containing alpha-1 proteinase inhibitor;

d) passing the eluted solution through a cation exchange resin;

e) collecting a flow-through from the cation exchange resin that contains alpha-1 proteinase inhibitor; and

f) contacting the eluted solution of step c) or the flow-through of step e) with a hydrophobic adsorbent of at least one HIC medium.

In some aspects, the present invention provides a method of purifying alpha-1 proteinase inhibitor in an aqueous solution comprising alpha-1 proteinase inhibitor and other proteins, the method comprising:

a) adjusting the pH, ionic strength, and protein concentration of the aqueous solution;

b) passing the solution through an ion exchange resin and collecting a flow-through that contains alpha-1 proteinase inhibitor; and

c) contacting the flow-through of step b) with a hydrophobic adsorbent of at least one HIC medium.

In other aspects, the present invention provides purified alpha-1 proteinase inhibitor and/or compositions comprising alpha-1 proteinase inhibitor, wherein the alpha-1 proteinase inhibitor is purified using methods described by the present invention.

### DETAILED DESCRIPTION

### Definitions

The term “adsorbent,” as used herein refers to at least one molecule affixed to a solid support, or at least one molecule that is, itself, a solid, which is used to perform chromatography, such as hydrophobic interaction chromatography. In the context of hydrophobic interaction chromatography, the adsorbent is a hydrophobic functional group.

“Chromatography” is the separation of chemically different molecules in a mixture from one another by contacting the mixture with an adsorbent, wherein one class of molecules reversibly binds to or is adsorbed onto the adsorbent. Molecules that are least strongly adsorbed to or retained by the adsorbent are released from the adsorbent under conditions where those more strongly adsorbed or retained are not.

Any or all chromatographic steps of the present invention can be carried out by a variety of mechanical techniques (also referred to as methods or procedures). Chromatography may be carried out, for example, in a column. The column may be run with or without pressure and from top to bottom or bottom to top. The direction of the flow of fluid in the column may be reversed during the chromatography process. Chromatography may also be carried out using a batch process in which the solid media is separated from the liquid used to load, wash, and elute the sample by any suitable method, including gravity, centrifugation, or filtration. Chromatography may also be carried out by contacting the sample with a filter that absorbs or retains some molecules in the sample more strongly than others.

Thus, although the various embodiments of the present invention may be described in the context of chromatography carried out in a column, for example, it is understood, however, that use of a column is merely one of several chromatographic modalities that may be used, and the illustration of the present invention using a column does not limit the application of the present invention to column chromatography, as those skilled in the art may readily apply the teachings to other modalities as well, such as those using a batch process or filter.

“Hydrophobic interaction chromatography (HIC)” is chromatography that utilizes specific reversible hydrophobic interactions of biomolecules in an aqueous salt solution as a basis for protein separation.

The term “contaminant,” as used herein refers to any foreign or objectionable molecule, particularly a biological macromolecule such as a DNA, an RNA, or a protein, other than the target protein being purified that is present in a sample of a target protein being purified. Contaminants include, for example, undesired proteins in a biological fluid, host cell proteins from cells used to express the target protein being purified, proteins that are part of an absorbent used in an affinity chromatography step that may leach into a sample during prior affinity chromatography step, and misfolded variants, dimers, or aggregates of the target protein itself.

The present invention provides a novel method for preparing α-1 PI, wherein the method includes an orthogonal purification step involving hydrophobic interaction chromatography (HIC) to provide surprisingly high over all yield and purity of α-1 PI product.

In one aspect, the present invention provides a method of purifying alpha-1 proteinase inhibitor in an aqueous solution comprising alpha-1 proteinase inhibitor and other proteins, the method comprising:

a) adjusting the pH, ionic strength, and protein concentration of the aqueous solution;

b) passing the solution through an ion exchange resin and collecting a flow-through that contains alpha-1 proteinase inhibitor; and

c) contacting the flow-through of step b) with a hydrophobic adsorbent of at least one HIC medium.

In another aspect, the present invention provides a method of purifying α-1 PI in an aqueous solution comprising α-1 PI and other proteins. The method comprises:

a) adjusting the pH, ionic strength, and protein concentration of the aqueous solution so that active α-1 PI does not bind to an ion exchange resin but other proteins in the solution do bind;

b) passing the solution through the ion exchange resin and collecting a flow through solution that contains α-1 PI; and

c) contacting the flow through solution of step b) with a hydrophobic adsorbent of at least one HIC medium. In one embodiment, the ion exchange resin is a strong ion exchange resin.

In one embodiment, steps a) and b) provide for purifying α-1 PI from aqueous protein-containing solutions by flow-through chromatography on cation exchange chromatography media under conditions of pH, ionic strength and protein concentration sufficient to assure that active α-1 PI does not bind to the media (or ion exchange resin) while other proteins, including inactive (or denatured) α-1 PI do bind to the media (or ion exchange resin). The influences of pH, ionic strength, and protein concentration on the binding of α-1 PI to a cation exchange resin are set forth in U.S. Pat. No. 5,610,285, the entire disclosure of which is hereby incorporated by reference herein. Also, ion exchange chromatography is described in U.S. Pat. No. 6,462,180, the entire disclosure of which is hereby incorporated by reference herein.

In preferred embodiments, the method includes the following steps: (1) the protein solution is dialyzed or diafiltered to an ionic strength of about 0.1 to 10 mmho/cm; (2) the solution pH is adjusted to about ≦6.0; (3) the protein solution is adjusted to about ≦10 mg protein/mL; (4) the solution is passed through a cation exchange chromatography resin; and (5) the flow through fraction (e.g., the flow through solution of step (b) above) of the chromatography is collected as purified α-1 PI.

The procedure is versatile enough that the cation chromatography will work on any number of starting materials, ranging from Cohn Fraction Effluent II+III, Cohn Fraction IV-1 paste (a presently preferred starting material) and purified α-1 PI, and still yield a substantially purified product.

As an option for large scale production of pharmaceutical product, a variety of additional steps, including viral inactivation may be added to optimize yield, improve viral safety, and assure regulatory compliance. These steps may include but are not limited to:

(1) Initial chromatography on a weak ion exchange resin (DEAE);
(2) Initial chromatography on a strong anion exchange resin (QAE);
(3) Viral inactivation utilizing either dry heat or pasteurization in solution;
(4) Viral exclusion filtration to remove possible viral contaminants;
(5) Chemical treatment such as solvent detergent treatment for viral inactivation; and
(6) Precipitation steps to partially purify starting material prior to chromatography.

In one embodiment, the method further comprises performing steps a) and b) more than once, wherein a viral inactivation step is performed on the solution prior to a final step a).

### HIC

Generally, HIC is a method for separating proteins based on the strength of their relative hydrophobic interactions with a hydrophobic adsorbent. Hydrophobicity is generally defined as the repulsion between a non-polar compound and a polar environment, typically aqueous solutions. Hydrophobic “interactions” are essentially the tendency of a polar environment to exclude non-polar (i.e., hydrophobic) compounds from the polar environment and force aggregation of the hydrophobic components amongst themselves. Accordingly, α-1 PI purification involving HIC can be based on the specific properties of α-1 PI's relative lack of hydrophobic interaction with a hydrophobic adsorbent.

In accordance with the present invention, HIC can be employed using a “flow through protocol,” where contaminant(s), but not α-1 PI, in a solution can be forced to bind adsorptively or to aggregate with hydrophobic functional groups (the adsorbent) affixed to a solid support.

### A. Flow-Through Protocol

In one embodiment, the ion exchange resin flow through solution containing α-1 PI can be contacted with the hydrophobic adsorbent under a condition sufficient to effect binding of the contaminants (or as much of the contaminants as possible) to the adsorbent, while α-1 proteinase inhibitor (and as few contaminants as possible) does not bind and flows through as a HIC flow-through fraction. In one embodiment, the solution is an eluted solution from an anion exchange resin, wherein the eluted solution comprises α-1 PI. In another embodiment, the solution is a cation exchange flow through solution, wherein the cation exchange flow through solution comprises α-1 PI.

Generally, HIC is performed by loading a solution containing α-1 PI onto the HIC column in an aqueous solution comprising a buffer and/or a salt. Suitable buffers include, but are not limited to citrate, succinate, phosphate, MES, ADA, BIS-TRIS Propane, PIPES, ACES, imidazole, diethylmalonic acid, MOPS, MOPSO, TES, TRIS buffer such as TRIS-HCl, HEPES, HEPPS, TRICINE, glycine amide, BICINE, glycylglycine, acetate, and borate buffers. Acceptable salts may include, but are not limited to sodium chloride, ammonium chloride, potassium chloride, sodium acetate, ammonium acetate, sodium sulfate, ammonium sulfate, ammonium thiocyanate, sodium citrate, sodium phosphate, and potassium, magnesium, and calcium salts thereof, and combinations of these salts.

In one embodiment, the salts include, but are not limited to, sodium citrate, sodium chloride, ammonium sulfate, sodium sulfate, and sodium phosphate, Acceptable ranges of salt concentrations used can be in the range of from 0 to about 2M sodium citrate, 0 to about 4M sodium chloride, 0 to about 3M ammonium sulfate, 0 to about 1M sodium sulfate and 0 to about 2M sodium phosphate. Other buffers and salts can also be used.

After loading, the adsorbent can be washed with more of the same loading solution (sans protein) to cause any α-1 proteinase inhibitor that is unbound to the adsorbent to flow through the adsorbent (a chase).

The α-1 proteinase inhibitor is then collected in the HIC flow-through fraction and chase. Conditions for binding contaminants, but not α-1 proteinase inhibitor, can be optimized by those skilled in the art without undue experimentation. The salt concentrations discussed herein are exemplary, and other salts and salt concentrations can be used by varying one or more parameters including, for example, pH, flow rates, and temperatures as is known in the art.

Because pH can directly effect the charge (i.e. hydrophobic properties of a protein in solution) adjustment of pH may impact the amount of salt necessary in the buffer. The pH under which HIC is performed can be varied. For example, the pH range may be between about 6.0 and about 8.0, or alternatively between about 6.5 and about 7.5. However, a broader range may be possible.

### B. HIC Media

Preferably, an HIC media comprises a support and the adsorbent affixed to the support. The HIC support can comprise a resin matrix prepared by any suitable method known to those skilled in the art. In general, the support may be of any material that is compatible with protein separations. Further, the support has been, or can be, modified by covalent linkage to the hydrophobic functional group thereby providing a HIC media (the support and adsorbent affixed to the support).

In one embodiment, the hydrophobic functional group is an alkyl (e.g., a C₂ to C₆ (or C₈ up to C₁₀). In another embodiment, the hydrophobic functional group is a cyclic, polycyclic, or heterocyclic aromatic functional group such as, for example, a phenyl group. One of ordinary skill in the art knows that the hydrophobicity may be adjusted by increasing the degree of substitution or density of the functional group on the support. In some embodiments, the functional group is selected from the group consisting of phenyl, octyl, propyl, alkoxy, butyl, and isoamyl.

Suitable supports may be any currently available or later developed materials having the characteristics necessary to practice the claimed method, and may be based on any synthetic, organic, or natural polymers. For example, commonly used support substances include organic materials such as cellulose, polystyrene, agarose, sepharose, polyacrylamide polymethacrylate, dextran and starch, and inorganic materials, such as charcoal, silica (glass beads or sand) and ceramic materials. Suitable solid supports are disclosed, for example, in Zaborsky “Immobilized Enzymes” CRC Press, 1973, Table IV on pages 28-46, which is incoporated herein by reference for its teaching of a solid support.

In one embodiment, the HIC medium comprises an average bead sizes of about 30 to about 100 microns, functional group densities of about 5 to about 50 micromoles per ml gel, and beads containing 4-6% crosslinked agarose. Also, HIC media are commercially available such as, for example, TSK-GEL Ether-5PW, Phenyl-5PW, and Butyl-NPR resin-based columns (Sigma-Aldrich, St. Louis, Mo.).

In some embodiments, it may be necessary to activate the support so that it will react with the hydrophobic functional group thereby forming the HIC media. Therefore, it is to be understood that if the source material for the support, for example agarose, is not itself amenable to reaction with a particular functional group, it may be conditioned or activated so that it will be amenable to such reactions.

Prior to equilibration and chromatography, the HIC media (the support and adsorbent affixed to the support) may be pre-equilibrated in a chosen solution, e.g. a salt and/or buffer solution. Pre-equilibration can serve the function of displacing a solution used for regenerating and/or storing the HIC medium. One of skill in the art will realize that the composition of the pre-equilibration solution depends on the composition of the storage solution and the solution to be used for the subsequent chromatography. Thus, appropriate pre-equilibration solutions may include the same buffer or salt used for performing the HIC. Optionally, the pre-equilibration solution may include buffer or salt at a higher concentration than is used to perform HIC.

Before a solution comprising α-1 proteinase inhibitor is applied to the HIC media, the HIC media can be equilibrated in the buffer or salt that will be used to dissolve or suspend the α-1 proteinase inhibitor. In some embodiments, equilibration may take place in a solution comprising sodium citrate between about 1 and about 20 millimolar and ammonium sulfate between about 500 and about 1000 millimolar. Equilibration may take place at pHs between about 6.0 and about 8.6, preferably at pHs between about 6.5 and 7.5. In one embodiment, the equilibration solution comprises a sodium citrate buffer at a concentration of about 10 millimolar, ammonium sulfate concentration of about 850 millimolar and at a pH of about 7.0.

Optionally, the HIC medium (e.g., HIC column) may be regenerated. For example, following HIC purification of α-1, any contaminants that may remain bound to the adsorbent may be released by stripping the HIC medium using a solution comprising the buffer or salt used for chromatography, but at a lower ionic strength to release the contaminant proteins. Then, the column may be regenerated using a solution that will have the effect of releasing most or all proteins from the chromatography medium and reducing or eliminating any microbial contamination that may be present in the chromatography medium. In one embodiment, such a solution may comprise sodium hydroxide. Other reagents can also be used. Subsequently, the column may be rinsed and stored in a solution that can discourage microbial growth. Such a solution may comprise sodium hydroxide, but other reagents can also be appropriate.

α-1 proteinase inhibitor, as well as contaminating proteins that may be present in a solution comprising α-1 PI, can be monitored by any appropriate method. Preferably, the technique should be sensitive enough to detect contaminants in the range between about 2 parts per million (ppm) (calculated as nanograms per milligram of the protein being purified) and 500 ppm. For example, enzyme-linked immunosorbent assay (ELISA), a method well known in the art, may be used to detect contamination.

In one embodiment, contamination of a solution comprising α-1 PI by protein contaminants can be reduced after HIC, preferably by at least about two-fold, illustratively, by at least about three-fold, by at least about five-fold, by at least about ten-fold, by at least about twenty-fold, by at least about thirty-fold, by at least about forty-fold, by at least about fifty-fold, by at least about sixty-fold, by at least about seventy-fold, by at least about 80-fold, by at least about 90-fold, and by at least about 100-fold.

In another embodiment, contamination of a solution comprising α-1 PI by such other protein contaminants after HIC is not more than about 10,000 ppm, preferably not more than about 2500 ppm, more preferably not more than about 400 ppm, more preferably not more than about 360 ppm, more preferably not more than about 320 ppm, more preferably not more than about 280 ppm, more preferably not more than about 240 ppm, more preferably not more than about 200 ppm, more preferably not more than about 160 ppm, more preferably not more than about 140 ppm, more preferably not more than about 120 ppm, more preferably not more than about 100 ppm, more preferably not more than about 80 ppm, more preferably not more than about 60 ppm, more preferably not more than about 40 ppm, more preferably not more than about 30 ppm, more preferably not more than about 20 ppm, more preferably not more than about 10 ppm, and most preferably not more than about 5 ppm. Such contamination can range from undetectable levels to about 10 ppm or from about 10 ppm to about 10,000 ppm.

In another aspect, the present invention provides a method of purifying α-1 PI from an aqueous solution containing α-1 PI, wherein the method comprises:

(a) removing a portion of contaminating proteins from the aqueous solution by precipitation in order to obtain a purified solution containing α-1 PI;

(b) passing the purified solution through an anion exchange resin so that α-1 PI binds to the anion exchange resin;

(c) eluting α-1 PI from the anion exchange resin to obtain an eluted solution containing α-1 PI;

(d) passing the eluted solution through a cation exchange resin;

(e) collecting a flow-through solution from the cation exchange resin that contains α-1 PI; and

(f) contacting the flow through solution of step e) with a hydrophobic adsorbent of at least one HIC medium.

In one embodiment, the steps a)-f) above are performed in order in the sequence recited. HIC is as described above.

In one embodiment, the aqueous solution containing α-1 PI is Cohn Fraction IV-1, wherein a portion of contaminating proteins from the Cohn Fraction IV-1 is removed by precipitation in order to obtain a purified solution containing α-1 PI. For example, the purification of α-1 PI can begin with Fraction IV-1 paste, as obtained through the Cohn-Oncley fractionation procedure for human plasma. See, e.g., E. J. Cohn, et al., J. Amer. Chem. Soc., 68, 459 (1946); E. J. Cohn, U.S. Pat. No. 2,390,074; and Oncley, et al., J. Amer. Chem. Soc., 71, 541 (1949) the entire disclosures of which are hereby incorporated by reference herein. The Cohn-Oncley process involves a series of cold ethanol precipitation steps during which specific proteins are separated according to isoelectric point by adjusting pH, ionic strength, protein concentration, temperature and ethanol concentration. The Fraction IV-1 paste obtained by this procedure is dissolved in a buffer solution and heated to activate α-1 PI. An initial purification step includes the precipitation of contaminating proteins and lipids from the dissolved Fraction IV-1. The α-1 PI is then precipitated from the dissolved Fraction IV-1 solution, and the crude α-1 PI is passed through an anion exchange resin to remove contaminating proteins. A viral inactivation is accomplished by pasteurization for 10 hours at 60° C. in a sucrose solution. Following pasteurization, the α-1 PI is diafiltered, bulked in NaCl/Na₃PO₄, sterile filtered, and lyophilized. Fraction IV-1 is typically a paste that can be dissolved in Tris buffer at a pH of about 9.25 to about 9.5 at about 40° C. A salt, such as sodium chloride (NaCl) may also be added to the solution.

Other fractions, such as Cohn Fraction II+III, for example, also can be used as a starting material. In this fraction α-1 PI is already dissolved in an aqueous solution. In one embodiment, the method includes the removal of at least a portion of contaminating proteins from a starting solution containing α-1 PI. Such contaminating proteins may include fibrinogen and albumin, for example. The portion of contaminating proteins is preferably removed by precipitation with a polyalkylene glycol, such as polyethylene glycol (PEG) or polypropylene glycol (PPG), for example. Other alcohols that are known to those of skill in the art to have similar properties may be used. PEG, the preferred polyalkylene glycol for use in methods of the invention, has a molecular weight of between about 2,000 and about 10,000, and preferably has a molecular weight of between about 3,000 and about 4,000. The PEG added to the solution is at least about 2% weight per volume of the mixture formed, is preferably about 3% to 15%, and is most preferably 11.5%. The pH of the solution may also be adjusted to precipitate the contaminating proteins. The pH is typically adjusted to between about 5.0 and about 6.0. The pH of the solution is adjusted by addition of an acid, such as acetic acid. The precipitate may then be separated from the solution by filtration, centrifugation, or any other conventional methods known in the art, to obtain a filtrate containing α-1 PI.

In one embodiment, the removing step a) comprises the steps of: (i) precipitating the portion of contaminating proteins from the aqueous solution; and (ii) separating the precipitated portion of contaminating proteins from the aqueous solution, thereby obtaining the purified solution containing α-1 PI. In another embodiment, the precipitating step comprises the steps of: (i) adding a polyalkylene glycol to the aqueous solution; and (ii) adjusting the pH of the aqueous solution to from about 5.0 to about 6.0. In some embodiments, the polyalkylene glycol is polyethylene glycol. The conductivity of the filtrate is then adjusted prior to passing the filtrate over an anion exchange resin. The equilibrium between an ion exchange resin and a protein solution is influenced by the ionic strength of the solution (see, e.g., Yamamato, et al., Biotechnol. Bioeng., 25:1373-91 (1983)). The conductivity of the filtrate is therefore adjusted so that the α-1 PI in the filtrate will bind to an anion exchange resin. This conductivity is typically between about 2.0 mS and 6.0 mS when measured at 25° C., but other ranges of conductivity may be necessary to bind the α-1 PI to an anion exchange resin. The conductivity is preferably adjusted by dilution of the filtrate, and not by gel filtration, diafiltration, or other methods of salt removal. The filtrate is preferably diluted with water, which may contain sodium phosphate (Na₃PO₄), or other buffers capable of providing a pH of about 6-7.

In one embodiment, the method further comprises the step of adjusting a pH of the purified solution to between about 6.25 and about 7.25 prior to passing the purified solution through the anion exchange resin.

After dilution of the filtrate, the solution can be applied directly to an anion exchange resin. Unlike known methods, the filtrate is not subjected to further PEG precipitation or diafiltration prior to chromatographic separation. The diluted filtrate is passed over an anion exchange resin, which is preferably a quaternary aminoethyl (QAE) resin. While QAE chromatography is preferred, other anion exchange resins, such as trimethylamino ethane (TMAE) and diethyl aminoethyl (DEAE), may be used in methods of the invention. The α-1 PI binds to the anion exchange resin. In a preferred embodiment, the anion exchange resin is washed with a buffer solution, such as Na₃PO₄ buffer, to remove another portion of contaminating proteins. For example, where the source of α-1 PI is plasma or a fraction thereof, the proteins typically removed are albumin and transferrin. During the buffer wash, α-1 PI remains bound to the anion exchange resin. After the buffer wash, α-1 PI is eluted from the anion exchange resin. Ceruloplasmin remains bound to the column during both the wash and elution.

In one embodiment, the method further comprises the step of, prior to the eluting step e), washing the anion exchange resin with a buffer solution to remove a portion of contaminating proteins from the anion exchange resin so that α-1 PI remains bound to the anion exchange resin. In one embodiment, the eluted solution from the anion exchange resin is passed through the cation exchange resin to obtain the cation exchange resin flow through comprising the α-1 PI. The pH, conductivity, and protein concentration of the eluted solution from the cation exchange resin are adjusted as described above.

In other embodiments, the method further comprises the step of removing viruses from the aqueous solution. Viral inactivation and/or viral removal also play a part in the purification of α-1 PI from aqueous solutions, such as plasma, for example. Known processes for the purification of α-1 PI utilize a dry heat treatment for the inactivation of viruses. This treatment can denature α-1 PI protein, however, thereby reducing the yield and/or purity of the α-1 PI. The methods of the invention deactivate and remove viruses without this heat treatment step, thereby increasing both yield and purity of α-1 PI obtained.

The above-described precipitation of contaminating proteins with 11.5% PEG also serves as one of the virus removal steps. Precipitation with 11.5% PEG removes both enveloped and non-enveloped viruses from the blood plasma fraction. This precipitation removes, with a ≧4 logs of clearance, at least four viruses, including HIV-1, BVDV, PRV, and Reovirus Type 3. In comparison, the dry heat process of known methods only results in ≧4 logs of clearance of three of these viruses; the Reovirus Type 3 is only removed at 1 log clearance. Additionally, the 11.5% PEG precipitation step has been shown to result in ≧4 logs of clearance of transmissible spongiform encephalopathies (i.e., TSE prions) from the blood plasma fraction. (See U.S. Pat. No. 6,437,102 entitled Method of Separating Prions from Biological Material).

Optionally, another viral deactivation is accomplished by addition of a non-ionic detergent. This step is preferably taken prior to passing the solution through the anion exchange resin. Non-ionic detergents for use in methods of the invention include, but are not limited to, Tweens, such as Tween 20 and Tween 80. Tween 20 is the preferred non-ionic detergent for use in methods of the invention. Tween 20 may be added at from about 0.33% to about 10% weight per volume of resulting mixture. Tween 20 is preferably added in the range of about 0.5% to about 2.0% and is most preferably added at 1.0%. Optionally, a solvent such as Tri-N-butyl-phosphate (TNBP) in a range of 0.01% to about 0.5% may be added along with the Tween 20 to increase effectiveness of the virus inactivation. The detergent treatment with 1% Tween 20 (Tween 20 plus TNBP) reduces enveloped viruses by >4 logs of clearance.

In one embodiment, the viral inactivation step comprises the steps of: (a) adding a detergent to the purified solution to obtain a mixture of detergent and purified solution; and (b) adjusting the pH of the mixture to from about 6.5 to about 8.5. Another embodiment of the invention optionally includes virus removal. Both enveloped and non-enveloped viruses can be removed by filtration, preferably by nanofiltration, or any other filtration methods known in the art. In a preferred embodiment, the solution obtained from the ion exchange resin, which includes α-1 PI, is subjected to nanofiltration. Nanofiltration reduces both enveloped and non-enveloped viruses by >4 logs of clearance. In one embodiment, the method further comprises the step of removing viruses from the aqueous solution. In another embodiment, the viral removal step comprises filtering the aqueous solution by nanofiltration. The methods of the current invention, therefore, preferably include two >4 logs of clearance steps for the removal of enveloped viruses and non-enveloped viruses.

Practice of the invention will be understood more fully from the following examples, which are presented herein for illustration only and should not be construed as limiting the invention in any way.

### EXAMPLES

### Example 1

In one embodiment, the starting material is Cohn Fraction IV-1 paste, which is obtained by the Cohn-Oncley fractionation technique, well known to those of skill in the art. The preparation of an aqueous solution from the Fraction IV-1 paste is described below.

The IV-1 paste is dissolved in 24 volumes of Tris buffer (IV-1 paste weight in kg times 24) between 20 and 8° C. The solution is mixed for approximately 4.5 hrs while maintaining the temperature between 2° and 8° C. After mixing, the pH of the solution is adjusted to between 9.25 and 9.5 using 1.0 M NaOH, which is added at a rate of 1.25 1/min. This solution is then mixed for 1 hr and the pH readjusted, if necessary. The solution is then heated to 39° C. to 41° C. for about 60 to about 90 minutes to dissolve the Fraction IV-1 paste in the buffer solution.

### Example 2

Fraction IV-1, like other plasma fractions, contains various proteins, such as lipoproteins, immunoglobulins, globulin, metaprotein, etc. These proteins must be separated from the α-1 PI, but some will also bind to an ion exchange resin and thereby interfere with the purification of α-1 PI. Before adding the solution to an anion exchange resin, therefore, a portion of these contaminating proteins is preferably removed first. This example describes one purification step in the process for the removal of contaminating proteins.

The Cohn Fraction IV-1 dissolved in the Tris buffer solution, as described above, is again cooled to between 2° C. and 8° C. To this cooled solution is added NaCl to 0.11 M. To this solution is then added 11.5% PEG MW 3,350 (Carbowax™, Union Carbide, Danbury, Conn.; suspension weight in kg times 0.115). The pH of the solution is then adjusted to between 5.10 and 5.35 with 1.0 M glacial acetic acid. A precipitate of contaminating proteins and viruses, including prion proteins, is formed. The solution is then centrifuged to remove precipitate followed by depth filtration such as a Cuno CP50 or SP90 (Cuno, Meriden, Conn.). Other depth filters may be used for this step. The paste obtained by the centrifugation and filtration is discarded. The filtrate contains the α-1 PI in PEG. Alternatively, the precipitate may be removed by filtration alone with or without the addition of filter aid, such as 2.5% of Hyflo Supercel™ (Celite Corporation, Lompoc, Calif.).

### Example 3

In a preferred embodiment, the filtrate obtained from the PEG precipitation outlined in Example 2 above is subjected to viral inactivation in a non-ionic detergent or a combination of solvent and non-ionic detergent. The pH of the filtrate from Example 2 above is adjusted to 7.0 to 7.2 with 1.0 M NaOH. To this solution is added Tween 20 to 1% (PEG filtrate weight in kg times 10.1 g/kg) or Tween 20 to 0.5% and TNBP to 0.03% (PEG filtrate weight in kg times 5.1 g/kg and 0.01 g/kg respectively). The pH is adjusted to 6.9 to 7.1 with 1.0 M NaOH. This solution is held between 20° C. and 30° C. for 6-10 hrs. This treatment reduces enveloped viruses in the solution containing α-1 PI by >4 logs of clearance.

### Example 4

The solution containing α-1 PI in both PEG and Tween 20 is then passed through an anion exchange resin to further purify the α-1 PI and separate it from the PEG and Tween 20. Prior to addition of the solution to the anion exchange resin, the conductivity of the solution is adjusted so that the α-1 PI will bind to the anion exchange resin. The solution resulting from Example 3 above is diluted with water-for-injection (WFI) until the conductivity of the solution is reduced to a value between about 2.0 mS and about 6.0 mS at 25° C. Additional 1% Tween 20 may be included in the WFI to prolong the contact time of the solution with the detergent. The WFI may contain Na₃ PO₄ (20 mM) at a pH of 6.5.

A Q Sepharose™ (Amersham-Pharmacia Biotech, Upsala, Sweden) fast flow column is prepared and equilibrated with a 20 mM Na₃PO₄ solution at pH 6.5. The solution of α-1 PI in Tween 20 and PEG is then added to the column at a concentration of 8-12 mg of α-1 PI per milliliter of resin. The flow rate of the column is 125 cm/hr. The α-1 PI binds to the column, which is then washed with the 20 mM Na₃PO₄ solution at pH 6.5. The Na₃PO₄ buffer further removes contaminating proteins, such as albumin and transferrin, for example, from the column. An elution buffer of 0.025 M Na₃PO₄/0.1 M NaCl at pH 6.95-7.05 is passed through the column to remove the α-1 PI. The eluate, which contains the α-1 PI, is collected. Ceruloplasmin remains bound to the column until the NaCl strip.

The purification step, therefore, accomplishes four objectives: 1) separation of the α-1 PI from the PEG; 2) separation of the α-1 PI from the Tween 20; 3) purification of the α-1 PI; and 4) prolongation of the contact time of viruses in the solution with the Tween 20.

### Example 5

In a preferred embodiment of the invention, the aqueous solution containing α-1 PI is subjected to a further purification step following the Q Sepharose™ chromatography outlined in Example 4 above. The further purification is accomplished by cation chromatography.

A Macro Prep High S™ (BioRad Laboratories, Hercules, Calif.) column is prepared and equilibrated with a 20 mM Na₃PO₄/5 mM NaCl buffer at pH 5.45 to 5.54 until the column effluent consistently has a pH≦5.60. Prior to adding the eluate containing α-1 PI obtained from the method of Example 4 above to the cation column, it may be concentrated by ultrafiltration and diafiltration. Dry Na₃PO₄ and NaCl are then added to the resulting concentrate to a final concentration of 20 mM Na₃PO₄ and 5 mM NaCl. The pH of the resulting solution is then adjusted to approximately 5.50 with 1.0 M glacial acetic acid. This solution is then applied to the column at a ratio of 5 mg contaminants (e.g., albumin and IgA) per milliliter of resin. The α-1 PI does not bind to the resin, while the contaminants do bind to the resin. The α-1 PI is chased through the column with a 20 mM Na₃PO₄/5 mM NaCl buffer at pH 5.45 to 5.54 to obtain a solution containing α-1 PI.

### Example 6

To further remove contaminating viruses, a filtration step is preferably included in methods of the invention. To the solution containing α-1 PI obtained by the process of Example 5 above is added NaCl to 0.75 M and the pH is adjusted to 7.0 with NaOH. The solution is then concentrated on a Viresolve 70™ (Millipore, Bedford, Mass.) membrane using differential diafiltration with 0.75 M NaCl until the volume is reduced to 5%-20% of its original volume. α-1 PI is washed through the Viresolve 70™ membrane with 3-5 diafiltration volumes of 0.75 M NaCl.

Following the filtration, the resulting solution containing purified α-1 PI is concentrated by ultrafiltration and diafiltration. After diafiltration, the solution is concentrated, and the concentrated α-1 PI is formulated at about 55 mg of α-1 PI per milliliter of 20 mM Na₃PO₄ and 100 mM NaCl at pH 7.0. Other virus filters such as an Asahi Planova virus filter may replace the Viresolve filter.

### Example 7

The aqueous solution containing α-1 PI can be subjected to a further purification step following the virus filtration outlined in Example 6 above. The further purification is accomplished by HIC chromatography.

A GE Healthcare Octyl Sepharose 4FF media column is prepared by equilibrating with a buffer of 10 mM sodium citrate, 850 mM ammonium sulfate at pH 7.0. The aqueous solution containing α-1 PI is adjusted by adding 850 mM ammonium sulfate and adjusting the pH 7.0. The solution is then applied to the Octyl Sepharose column and any nonbound protein is washed out of the column with the equilibration buffer. The collected flow through and wash contained the purified α-1 PI.

The aqueous solution containing α-1 PI is then ultrafiltered and diafiltered to remove salts and prepared for final formulation. The formulated solution is sterile filtered. The resulting solution is lyophilized using methods known in the art.

### Example 8

TABLE 1
Alpha-1 Purity from Pre-clinical Pilot Lots

| | Alpha-1 Activity | Total Protein | Specific | % Monomer by |
|---|---|---|---|---|
| Batch | mg/ml⁽¹⁾ | mg/ml⁽²⁾ | Activity⁽³⁾ | SEC HPLC⁽⁴⁾ |
| T06Au01 | 57.3 | 56.2 | 1.02 | 95.5 |
| T06Au02 | 58.0 | 53.3 | 1.10 | 95.1 |
| T06Au03 | 61.2 | 56.8 | 1.08 | 91.5 |
| Average | 58.9 | 55.4 | 1.07 | 94.1 |
| N = 3 | | | | |

⁽¹⁾As described by Beatty, et al., JBC, 255:3931-3934, 1980, developed and validated for this matrix
⁽²⁾Standard Biuret Protein Assay, developed and validated for this matrix
⁽³⁾(Alpha-1 mg/ml)/(Total Protein mg/ml)
⁽⁴⁾Standard Size Exclusion HPLC, developed and validated for this matrix

The invention may be embodied in other specific forms without departing from the spirit or essential characteristics thereof. The foregoing examples are included by way of illustration only. Accordingly, the scope of the invention is limited only by the scope of the appended claims.

1. A method of purifying alpha-1 proteinase inhibitor in an aqueous solution comprising alpha-1 proteinase inhibitor and other proteins, the method comprising:
a) adjusting the pH, ionic strength, and protein concentration of the aqueous solution so that active alpha-1 proteinase inhibitor does not bind to an ion exchange resin but other proteins in the solution do bind;
b) passing the solution through the ion exchange resin and collecting a flow-through that contains alpha-1 proteinase inhibitor; and
c) contacting the flow-through of step b) with a hydrophobic adsorbent of at least one HIC medium.

2. The method of claim 1, wherein the hydrophobic adsorbent is a functional group selected from the group consisting of phenyl, octyl, propyl, alkoxy, butyl, and isoamyl.

3. The method of claim 1, wherein contacting occurs under a condition sufficient to effect binding of at least a portion of any remaining contaminants to the adsorbent, wherein alpha-1 proteinase inhibitor does not bind and flows through as a HIC flow-through fraction.

4. The method of claim 3, wherein the condition comprises a salt concentration of 10 mM sodium citrate and 850 mM ammonium sulfate.

5. The method of claim 1, wherein contacting occurs under a condition sufficient to effect binding of at least a portion of any remaining contaminants to the adsorbent, wherein alpha-1 proteinase inhibitor does not bind and flows through as a HIC flow-through fraction.

6. The method of claim 1, wherein the aqueous solution is Cohn Fraction IV-1.

7. The method of claim 1, wherein steps a) and b) are performed more than once and a viral inactivation step is performed on the solution prior to the final step a).

8. The method of claim 7, wherein the viral inactivation step comprises heating the alpha-1 proteinase inhibitor at greater than or equal to about 60° C. for greater than or equal to about 10 hours.

9. The method in claim 7, wherein the viral inactivation step comprises adding one or more chemical agent.

10. The method in claim 7, wherein the viral inactivation step comprises adding tri-n-butyl phosphate and a detergent to the solution.

11. A method of purifying alpha-1 proteinase inhibitor from an aqueous solution containing alpha-1 proteinase inhibitor, the method comprising:
a) removing a portion of contaminating proteins from the aqueous solution by precipitation in order to obtain a purified solution containing alpha-1 proteinase inhibitor;
b) passing the purified solution through an anion exchange resin so that alpha-1 proteinase inhibitor binds to the anion exchange resin;
c) eluting alpha-1 proteinase inhibitor from the anion exchange resin to obtain an eluted solution containing alpha-1 proteinase inhibitor;
d) passing the eluted solution through a cation exchange resin;
e) collecting a flow-through from the cation exchange resin that contains alpha-1 proteinase inhibitor; and
f) contacting the eluted solution of step c) or the flow-through of step e) with a hydrophobic adsorbent of at least one HIC medium.

12. The method of claim 11, wherein the aqueous solution containing alpha-1 proteinase inhibitor is Cohn Fraction IV-1.

13. The method of claim 11, wherein the hydrophobic adsorbent is a functional group selected from the group consisting of phenyl, octyl, propyl, alkoxy, butyl, and isoamyl.

14. The method of claim 11, wherein contacting occurs under a condition sufficient to effect binding of at least a portion of any remaining contaminants to the adsorbent, wherein alpha-1 proteinase inhibitor does not bind and flows through as a HIC flow-through fraction.

15. The method of claim 11, wherein the removing step comprises the steps of: (i) precipitating the portion of contaminating proteins from the aqueous solution; and (ii) separating the precipitated portion of contaminating proteins from the aqueous solution, thereby obtaining the purified solution containing alpha-1 proteinase inhibitor.

16. The method of claim 15, wherein the precipitating step comprises the steps of: (1) adding a polyalkylene glycol to the aqueous solution; and (2) adjusting the pH of the aqueous solution to from about 5.0 to about 6.0.

17. The method of claim 16, wherein the polyalkylene glycol is polyethylene glycol.

18. The method of claim 11, further comprising the step of, prior to the eluting step, washing the anion exchange resin with a buffer solution to remove a portion of contaminating proteins from the anion exchange resin so that alpha-1 proteinase inhibitor remains bound to the anion exchange resin.

19. The method of claim 11 further comprising a viral inactivation step.

20-29. (canceled)

30. A method of purifying alpha-1 proteinase inhibitor in an aqueous solution comprising alpha-1 proteinase inhibitor and other proteins, the method comprising:
a) adjusting the pH, ionic strength, and protein concentration of the aqueous solution;
b) passing the solution through an ion exchange resin and collecting a flow-through that contains alpha-1 proteinase inhibitor; and
c) contacting the flow-through of step b) with a hydrophobic adsorbent of at least one HIC medium.

31. (canceled)
